SEQUENCE LISTING (1 sequence)

SEQ ID NO: 1 - protein, 381 aa, Camelus dromedarius
  SOURCE 1..381: /mol_type=“protein” /organism=“Camelus dromedarius”
MRCLVVLLAALALSQASGITRIPLHKGKTLRKALKERGLLEDFLQRQQYAVSSKYSSLGKVAREPLTSYLDSQYFGKIYIGTPPQEFTVVFDTGSSDLWVPSIYCKSNVCKNHHRFDPRKSSTFRNLGKPLSIHYGTGSMEGFLGYDTVTVSNIVDPNQTVGLSTEQPGEVFTYSEFDGILGLAYPSLASEYSVPVFDNMMDRHLVARDLFSVYMDRNGQGSMLTLGAIDPSYYTGSLHWVPVTLQQYWQFTVDSVTINGVAVACVGGCQAILDTGTSVLFGPSSDILKIQMAIGATENRYGEFDVNCGNLRSMPTVVFEINGRDYPLSPSAYTSKDQGFCTSGFQGDNNSELWILGDVFIREYYSVFDRANNRVGLAKAI

(19) United States
(12) Patent Application Publication
(10) Pub. No.: US 2016/0002618 A1
(43) Pub. Date: Jan. 7, 2016

(54) IMPROVED PURIFICATION OF PROTEINS VIA A DEGLYCOSYLATION STEP

(71) Applicant: CHR. HANSEN A/S, Hoersholm (DK)

(72) Inventors: Jonas JACOBSEN, Copenhagen (DK); Hans BISGAARD-FRANTZEN, Roedovre (DK); Johannes Maarten VAN DEN BRINK, Herlev (DK); Jesper Langholm JENSEN, Copenhagen (DK); Sari Charlotte HANSEN, Hoersholm (DK)

(73) Assignee: Chr. Hansen A/S, Hoersholm (DK)

(21) Appl. No.: 14/765,453

(22) PCT Filed: Jan. 31, 2014
(86) PCT No.: PCT/EP2014/051931

§ 371 Date: Aug. 3, 2015

(30) Foreign Application Priority Data
Feb. 5, 2013 (EP) 13154063.5
Feb. 11, 2013 (EP) 13154797.8

(51) Int. Cl. C12N 9/64

(52) U.S. Cl. CPC C12N 9/6483; C12N 9/6478; C12Y 304/23004; C12Y 304/23023

(57) ABSTRACT

A method for purifying a polypeptide of interest by use of a deglycosylation step.

### FIELD OF THE INVENTION

The present invention relates to a method for purifying a polypeptide of interest by use of a deglycosylation step.

### BACKGROUND ART

Many times a protein purification protocol contains one or more chromatographic steps. An example of a procedure in chromatography is to flow the solution containing the protein through a column packed with various materials. Different proteins interact differently with the column material, and can thus be separated by the time required to pass the column, or the conditions required to elute the protein from the column.

Chromatography is the collective term for a set of laboratory techniques for the separation of mixtures. The mixture is dissolved in a fluid called the mobile phase, which carries it through a structure holding another material called the stationary phase. The various constituents of the mixture travel at different speeds, causing them to separate. The separation is based on differential partitioning between the mobile and stationary phases. Subtle differences in a compound's partition coefficient result in differential retention on the stationary phase and thus changing the separation.

Many different chromatographic methods exist.

Hydrophobic Interaction Chromatography (HIC) is based on a ligand comprising hydrophobic regions (e.g. a benzyl group). The hydrophobic part of the ligand attracts hydrophobic region on the proteins and the greater the hydrophobic region on the protein the stronger the attraction between the ligand and that particular protein.

Ion exchange chromatography separates compounds according to the nature and degree of their ionic charge. The column to be used is selected according to its type and strength of charge. Anion exchange resins have a positive charge (e.g. an amine) and are used to retain and separate negatively charged compounds, while cation exchange resins have a negative charge (e.g. a carboxylate group) and are used to separate positively charged molecules.

As known in the art—Mixed-mode chromatography (MMC), or multimodal chromatography, refers to chromatographic methods that utilize more than one form of interactions between the stationary phase and analytes in order to achieve their separation. What is distinct from conventional single-mode chromatography is that the secondary interactions in MMC cannot be too weak, and thus they also contribute to the retention of the solutes.

As example of a MMC ligand may e.g. be benzylamine, where benzyl may be seen as the hydrophobic part and the amine may be seen as the positive charge part (i.e. for e.g. anion exchange).

Glycosylation of proteins is a common post-translational modification in eukaryotes. Glycosylations can be either N-linked (attached to Asn) or O-linked (attached to Ser or Thr). The effect of glycosylation on enzymes can affect many of its properties such as solubility, hydrophobicity etc. and as such the opposite—so called deglycosylation—may also affect protein properties.

As known in the art—the term “glycosidase” (also called glycoside hydrolase) refers to an enzyme that catalyzes the hydrolysis of the glycosidic linkage/bond—a glycosidase may herein also be termed a deglycosylation enzyme.

Enzymatic coagulation of milk by milk-clotting enzymes, such as chymosin and pepsin, is one of the most important processes in the manufacture of cheeses. Enzymatic milk coagulation is a two-phase process: a first phase where a proteolytic enzyme, chymosin or pepsin, attacks K-casein, resulting in a metastable state of the casein micelle structure and a second phase, where the milk subsequently coagulates and forms a coagulum.

Chymosin (EC 3.4.23.4) and pepsin (EC 3.4.23.1), the milk clotting enzymes of the mammalian stomach, are aspartic proteases belonging to a broad class of peptidases.

WO01/58924A2 (Upfront Chromatography A/S, Denmark) describes use of the mixed mode ligand benzylamine for chromatographic purification of a milk-clotting enzyme such e.g. Chymosin.

WO01/58924A2 does not describe anything of herein significant relevance in relation to deglycosylation of the protein of interest before it is adsorbed/bound to the ligand in the purification process.

### SUMMARY OF THE INVENTION

A problem to be solved by the present invention is to provide a new method for purifying a polypeptide of interest, wherein one is able to obtain an increased amount of the polypeptide of interest (number of molecules).

As discussed herein and without being limited to theory—a herein relevant important technical teaching relates to that the present inventors have identified that by proper deglycosylation of a glycoprotein of interest one may get a better/higher binding capacity to ligands which comprise a hydrophobic part and/or a positively charged part.

As such the deglycosylation step is a routine step for the skilled person to perform—the skilled person knows a number of different glycosidase enzymes and knows how to add a suitable one to the sample in order to obtain the herein relevant deglycosylation of the polypeptide/protein of interest.

Further, the skilled person knows a number of different herein relevant ligands (see e.g. the review article: Yang et al, Journal of Chromatography A, 1218 (2011) 8813-8825).

The skilled person knows a number of herein relevant purification/separation techniques, which is based on adsorption/binding of a protein of interest to a ligand—examples are e.g. column chromatography, expanded bed adsorption (EBA), ion-exchange chromatography, etc.

In working examples herein can be seen that deglycosylation of two structurally different enzymes (mucorpepsin derived from Rhizomucor miehei and camel chymosin) gave significant better binding capacity to benzylamine ligands—i.e. a ligand that may be seen as comprising both an hydrophobic part (benzyl) and a positively charged part (amine).

In working examples herein can be seen that above mentioned positive improved binding capacity were also demonstrated for a ligand that may be seen as comprising only an hydrophobic part (see Example 3 herein) and a ligand that may be seen as comprising only a positively charged part (see Example 4 herein).

Without being limited to theory—it may be that the higher binding to e.g. the hydrophobic part of a ligand could be explained by increased affinity to the ligand as a result of loss of hydrophilic glycans.

Without being limited to theory—it may be that the higher binding to e.g. the positively charged part of a ligand could be explained by a change in local exposure of charges on the protein surface as a result of loss of glycans.

Accordingly, a first aspect of the invention relates to a method for purifying a polypeptide of interest from an aqueous medium comprising such a polypeptide of interest, wherein the method comprises the steps of:

(i): obtaining an aqueous sample consisting of a number of components including the polypeptide of interest in a glycosylated form;

(ii): adding a glycosidase and/or a chemical treatment (such as such as treatment with periodate) to the sample of step (i) in order to deglycosylate the polypeptide of interest to obtain an aqueous load medium;

(iii): applying the load medium of step (ii) onto a solid phase comprising a solid base matrix containing ligands which comprise a hydrophobic part and/or a positively charged part in order to obtain adsorption of the polypeptide of interest to the ligand;

(iv): eluting the polypeptide of interest from the solid phase in order to recover the polypeptide of interest and thereby obtaining the purified polypeptide of interest;

wherein the amount of the purified polypeptide of interest (number of molecules) obtained in step (iv) is at least 5% increased as compared to an identically performed comparative method for purifying the polypeptide of interest, which does not comprise the step (ii).

Preferably, there is in step (ii) of the first aspect added a glycosidase.

It is routine work for the skilled person to make an identically performed comparative method to test if the amount of the purified polypeptide of interest (number of molecules) obtained in step (iv) is at least 5% increased due to the addition of the glycosidase in step (ii).

One simply performs the method of the first aspect and also a comparative method, where everything is completely identical expect that the adding a glycosidase step (ii) is not used in the comparative method.

If the result is that one gets an amount of the purified polypeptide of interest (number of molecules) obtained in step (iv) that is at least 5% increased as compared to the identically performed comparative method (i.e. without step (ii)) then one has a situation, wherein the amount of the purified polypeptide of interest (number of molecules) obtained in step (iv) is at least 5% increased as compared to an identically performed comparative method for purifying the polypeptide of interest, which does not comprise the step (ii).

Preferably, the amount of the polypeptide of interest (number of molecules) obtained in step (iv) is at least 10% increased as compared to an identically performed comparative method for purifying the polypeptide of interest, which does not comprise the step (ii).

More preferably, the amount of the polypeptide of interest (number of molecules) obtained in step (iv) is at least 25% increased as compared to an identically performed comparative method for purifying the polypeptide of interest, which does not comprise the step (ii).

Even more preferably, the amount of the polypeptide of interest (number of molecules) obtained in step (iv) is at least 50% increased as compared to an identically performed comparative method for purifying the polypeptide of interest, which does not comprise the step (ii).

As known in the art—a suitable unit for number of molecules is the unit termed mole.

As understood by the skilled person in the art—a composition comprising the purified polypeptide of interest of step (iv) would have a different glycosylation profile—i.e. it could be considered a new composition as such.

Accordingly, a second aspect of the invention relates to a composition comprising purified polypeptide of interest, wherein the composition is obtainable by the method of the first aspect and embodiments thereof as described herein.

Without being limited to theory—it is believed that all commercial relevant products of milk clotting enzymes are produced by use of a eukaryotic production host cell or extracted from e.g. relevant animal stomach (e.g. bovine stomach) that glycosylate milk clotting enzyme of interest during the production/expression (e.g. recombinant production)—said in other words, all commercial relevant products of milk clotting enzymes are glycosylated enzymes.

Commercial relevant examples are e.g. a mucorpepsin derived from Rhizomucor miehei as described in e.g. EP0805866B1 (Harboe et al, Chr. Hansen A/S, Denmark), wherein a commercially relevant product Hannilase® (Chr. Hansen A/S) is produced by using Rhizomucor miehei as eukaryotic production host cell.

Another example is Camelius dromedarius chymosin as described in e.g. WO02/36752A2 (Chr. Hansen), wherein a commercially relevant product CHY-MAX® M (Chr. Hansen A/S) is produced by using Aspergillus niger as eukaryotic production host cell.

As discussed above and without being limited to theory—a herein relevant important technical teaching relates to that the present inventors have identified that by proper deglycosylation of a glycoprotein of interest one may get a better/higher binding capacity to ligands which comprise a hydrophobic part and/or a positively charged part.

Accordingly, based on the technical teaching herein one may say that the skilled person could routinely identify an alternatively method that the method of the first aspect as described herein to for purifying a commercial relevant milk clotting enzyme of interest, wherein the method essentially could be seen in using a production host cell which does not give significant glycosylation of the milk clotting enzyme.

This not significantly glycosylated milk clotting enzyme of interest may then by purified by applying it onto a relevant ligand as described herein in relation to the first aspect of the invention and embodiments thereof.

Accordingly, a separate aspect of the invention relates to a method for purifying a milk clotting enzyme of interest from an aqueous medium comprising such a milk clotting enzyme of interest, wherein the method comprises the steps of:

(I): producing the milk clotting enzyme of interest in a production host cell, wherein the production host cell does not give significant glycosylation of the milk clotting enzyme, to obtain an aqueous sample consisting of a number of components including at least 10 gram (weight dry matter) (such as preferably at least 100 gram or at least 1 kg weight dry matter) of the milk clotting enzyme of interest in an essentially not glycosylated form and thereby obtaining an aqueous load medium;

(II): applying the load medium of step (I) onto a solid phase comprising a solid base matrix containing ligands which comprise a hydrophobic part and/or a positively charged part in order to obtain adsorption of the polypeptide of interest to the ligand; and

(III): eluting the milk clotting enzyme of interest from the solid phase in order to recover the milk clotting enzyme of interest and thereby obtaining the purified milk clotting enzyme of interest.

As understood by the skilled person in the present context—a herein relevant example of a suitable production host cell of step (I) could e.g. be a prokaryotic production host cell (such as e.g. E. coli of Bacillus).

As known in the art—some eukaryotic production host cells may also be cells characterized by that do not give significant glycosylation of the protein of interests (here a milk clotting enzyme of interest). Suitable herein relevant examples of this are genetically engineered cells, wherein genes essential for glycosylated are inactivated (e.g. deleted or mutated).

As understood by the skilled person in the present context—suitable herein described relevant embodiments of the method of the first aspect may also be preferred embodiment for the method of the separate aspect described immediately above—for instance a preferred ligand may e.g. be benzylamine and/or a preferred milk clotting enzyme of interest may e.g. be mucorpepsin derived from Rhizomucor miehei; or

a chymosin, wherein the polypeptide sequence of the chymosin comprises a sequence, which has at least 90% sequence identity with the mature polypeptide of SEQ ID NO: 1 (Camel chymosin), which is from amino acid position 59 to amino acid position 381 of SEQ ID NO: 1.

### Definitions

All definitions of herein relevant terms are in accordance of what would be understood by the skilled person in relation to the herein relevant technical context.

The term “enzyme” refers to an enzyme with enzymatic activity—i.e. an active enzyme. For instance, when the enzyme is a milk-clotting enzyme (e.g. chymosin) the activity may be determined milk-clotting activity (C) expressed in International Milk-Clotting Units (IMCU). It is determined by a standard method (15011815 IDF157, 2007), that describes the ability to aggregate milk by cleaving the Phe105-Met106-bond or nearby bonds of κ-casein.

The term “glycan” refers to a polysaccharide or oligosaccharide. Glycans can be homo or heteropolymers of monosaccharide residues, and can be linear or branched. Glycan may also be used to refer to the carbohydrate portion of a glycoconjugate, such as a glycoprotein, glycolipid, or a proteoglycan.

The term “glycosidase” (also called glycoside hydrolase) refers to an enzyme that catalyzes the hydrolysis of the glycosidic linkage/bond—a glycosidic bond is a type of covalent bond that joins a carbohydrate (sugar) molecule to another group, which may or may not be another carbohydrate. A glycosidase that partially or completely deglycosylate N-linked glycans may herein be termed an N-linked glycosidase. Similarly, a glycosidase that partially or completely deglycosylate O-linked glycans may herein be termed an O-linked glycosidase.

The term N-linked glycosidase is a well-defined term in the art and the skilled person knows if a specific glycosidase of interests is a N-linked glycosidase or not.

Similarly, the term O-linked glycosidase is a well-defined term in the art and the skilled person knows if a specific glycosidase of interests is an O-linked glycosidase or not. A glycosidase may herein also be termed a deglycosylation enzyme.

The term “glycosylation” is the enzymatic process that attaches glycans to proteins, lipids, or other organic molecules.

The term “N-linked glycans” refers to glycans attached to a nitrogen of normally asparagine or arginine side chains.

The term “O-linked glycans” refers to glycans attached to the hydroxy oxygen of normally serine, threonine, tyrosine, hydroxylysine, or hydroxyproline side chains, or to oxygens on lipids such as ceramide.

The term “polypeptide” relates to a single linear polymer chain of amino acids bonded together by peptide bonds.

The term “polypeptide in a glycosylated form” are polypeptids that contain oligosaccharide chains (glycans) covalently attached to polypeptide side-chains. The carbohydrate is attached to the polypeptide in a cotranslational or posttranslational modification. This may herein alternatively herein be termed “glycopolypetides”.

The term “protein” relates to a relatively large biological molecules consisting of one or more chains of amino acids. As understood in the art—a protein is an example of a polypeptide.

The term “protein in a glycosylated form” are proteins that contain oligosaccharide chains (glycans) covalently attached to protein side-chains. The carbohydrate is attached to the polypeptide in a cotranslational or posttranslational modification. This may herein alternatively herein be termed “glycoproteins”.

The term “Sequence Identity” relates to the relatedness between two amino acid sequences or between two nucleotide sequences.

For purposes of the present invention, the degree of sequence identity between two amino acid sequences is determined according to the art and preferably determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment) For purposes of the present invention, the degree of sequence identity between two deoxyribonucleotide sequences is determined according to the art and preferably determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides×100)/(Length of Alignment−Total Number of Gaps in Alignment).

Embodiment of the present invention is described below, by way of examples only.

### DETAILED DESCRIPTION OF THE INVENTION

### A Polypeptide of Interest

Without being limited to theory—it is believed that the method of a first aspect may be relevant in relation to in principle any polypeptide of interest.

It may be preferred that the polypeptide of interest is a protein of interest. The protein of interest may be an enzyme of interest, such as e.g. a lipase, protease, amylase, cellulose, beta-galactosidase or a peroxidase.

As known in the art—enzymatic coagulation of milk by milk-clotting enzymes, such as chymosin and pepsin, is one of the most important processes in the manufacture of cheeses. Enzymatic milk coagulation is a two-phase process: a first phase where a proteolytic enzyme, chymosin or pepsin, attacks K-casein, resulting in a metastable state of the casein micelle structure and a second phase, where the milk subsequently coagulates and forms a coagulum.

Chymosin (EC 3.4.23.4) and pepsin (EC 3.4.23.1), the milk clotting enzymes of the mammalian stomach, are aspartic proteases belonging to a broad class of peptidases.

Another example of a milk-clotting enzyme is mucorpepsin (EC 3.4.23.23). In working example herein have been demonstrated that the method of the first aspect may be used to improve the purification of two different chymosins (one a mucorpepsin derived from Rhizomucor miehei and one chymosin derived from Camelius drome-daries). In a preferred embodiment, the enzyme of interest is an aspartic protease of interest.

In a preferred embodiment, the enzyme of interest is a milk-clotting enzyme of interest—preferably a milk-clotting enzyme selected from the group consisting of chymosin (EC 3.4.23.4), pepsin (EC 3.4.23.1) and mucorpepsin (EC 3.4.23.23).

In a preferred embodiment—the enzyme of interest is a mucorpepsin derived from Rhizomucor miehei as described in e.g. EP0805866B1 (Harboe et al, Chr. Hansen A/S, Denmark).

As said in paragraph [0021] of EP0805866B1—a herein relevant mucorpepsin derived from Rhizomucor miehei comprises in its active form 361 amino acid residues and has a calculated molecular weight excluding glycosyl moieties of around 38,701.

As explained in paragraph [0013] of EP0805866B1—a herein relevant mucorpepsin derived from Rhizomucor miehei is commercially sold as Hannilase® by Chr. Hansen A/S.

A herein relevant enzyme of interest is Camelius dromedarius chymosin as described in e.g. WO02/36752A2 (Chr. Hansen). It may herein alternatively be termed camel chymosin and the publically known amino acid sequence is shown in SEQ ID NO: 1 herein.

Accordingly, in a preferred embodiment the enzyme of interest is a chymosin, wherein the polypeptide sequence of the chymosin comprises a sequence, which has at least 90% (preferably at least 95%, more preferably at least 99%) sequence identity with the mature polypeptide of SEQ ID NO: 1 (Camel chymosin), which is from amino acid position 59 to amino acid position 381 of SEQ ID NO: 1.

Preferably, the enzyme of interest is a chymosin, wherein the polypeptide sequence of the chymosin comprises the mature polypeptide of SEQ ID NO: 1 (Camel chymosin), which is from amino acid position 59 to amino acid position 381 of SEQ ID NO: 1.

### Step (i) of First Aspect—Obtaining Sample With the Polypetide of Interest in a Glycosylated Form

Step (i) of first aspect relates to obtaining an aqueous sample consisting of a number of components including the polypetide of interest in a glycosylated form.

The sample may be a sample wherein the majority of the polypetide of interest is in a glycosylated form.

However, it may alternatively be a sample, wherein e.g. less than 50% of the polypetide of interest is in a glycosylated form.

It is routine work for the skilled person to obtain such a sample.

It may e.g. be obtained by recombinant production of e.g. a protein of interest in a eukaryotic production host cell—as known in the art a eukaryotic production host cell may glycosylate a protein of interest during the recombination production/expression.

Accordingly, by performing production (e.g. recombinant production) of e.g. a protein of interest in a eukaryotic production host cell one may obtain a sample consisting of a number of components including the polypetide/protein of interest in a glycosylated form as required in step (i).

As known in the art—before further downstream purification of e.g. the protein of interest one normally removes/separates production host cells and other unwanted material in the fermentation media (by e.g. centrifugation and/or filtrating)—i.e. to get a sample comprising the protein of interest without too many unwanted components such as e.g. production host cells. As known in the art—this may sometimes be termed a non-purified first filtrate—this term may be used herein and it may be an example of a herein relevant sample consisting of a number of components including the polypetide of interest in a glycosylated form of step (i).

Alternatively, it may be a more purified sample—e.g. a sample where one has made a first purification by use of e.g. Size-exclusion chromatography (SEC).

A number of suitable eukaryotic production host cells for production (e.g. recombinant production) of a e.g. a protein of interest are known in the art—herein relevant examples are e.g. mammalian cells (such as e.g. Chinese hamster ovary (CHO) cells) or fungal cells (such as e.g. Aspergillus cells—preferably Aspergillus niger or Aspergillus oryzae).

WO02/36752A2 (Chr. Hansen) describes a recombinant method to produce Camelius dromedarius chymosin (Camel chymosin) using Aspergillus cells (preferably Aspergillus niger) as production host cells.

Accordingly, when the enzyme of interest is a milk-clotting enzyme (e.g. selected from the group consisting of chymosin (EC 3.4.23.4), pepsin (EC 3.4.23.1) and mucorpepsin (EC 3.4.23.23)) it may be preferred that the recombinant production host cell is an Aspergillus cell (preferably preferably Aspergillus niger).

Mucorpepsin derived from Rhizomucor miehei may preferably be produced by use of Rhizomucor miehei as production host cell.

### Step (ii) of First Aspect—Adding a Glycosidase to the Sample of Step (i)

Step (ii) of first aspect relates to adding a glycosidase to the sample of step (i) in order to deglycosylate the polypetide (e.g. protein) of interest to obtain an aqueous load medium.

The term “load medium” simply relates to the medium obtained in step (ii) and which is then used in step (iii) of the first aspect.

The load medium may be a load medium wherein the majority of the glycopolypetides of the sample of step (i) have been deglycosylated.

Alternatively, load medium may be a load medium wherein e.g. less than 50% of the glycopolypetides of the sample of step (i) have been deglycosylated.

In the present context—this step (ii) may be seen as a key step and it is linked to a main objective/advantage of the method of the first aspect, which is to improve the purification method as such and thereby getting an increased amount of the purified polypeptide/protein of interest.

As discussed above and without being limited to theory—a herein relevant important technical teaching relates to that the present inventors have identified that by proper deglycosylation of a glycoprotein of interest one may get a better/higher binding capacity to ligands which comprise a hydrophobic part and/or a positively charged part.

As such this step is a routine step for the skilled person to perform—the skilled person knows a number of different glycosidase enzymes and knows how to add a suitable one to the sample in order to obtain the herein relevant deglycosylation of the polypeptide/protein of interest.

Further—by making a relatively limited amount of e.g. trial/error experiments the skilled person may identify a preferred good glycosidase enzyme in relation to a specific polypeptide/protein of interest—i.e. a glycosidase enzyme which in the present context properly can deglycosylate the polypeptide/protein of interest.

It is also routine work for the skilled person to identify a suitable/optimal amount of active glycosidase added in step (ii) in order to get a herein relevant deglycosylation of the polypeptide/protein of interest.

The last paragraph of the method of the first aspect relates to that the amount of the purified polypeptide of interest (number of molecules) obtained in step (iv) is at least 5% increased as compared to an identically performed comparative method for purifying the polypeptide of interest, which does not comprise the step (ii).

As understood by the skilled person—this paragraph implies a limitation to step (ii) of the first aspect—in the sense that if e.g. a not active or a not working glycosidase is used in step (ii) then one will not obtain the 5% increase of the amount of the purified polypeptide of interest.

It is also contemplated that deglycosylation in step (ii) of the first aspect may be obtained in a by providing a production host cell that in addition to the polypeptide of interest expresses a glycosidase enzyme (such as e.g. Endo-H) whereby the initially glycosylated polypeptide of interest is deglycosylated intracellularly or following secretion.

Accordingly, an embodiment of the invention may be:
wherein the sample consisting of a number of components including the polypetide/protein of interest in a glycosylated form as required in step (i) of the first aspect is obtained by production (e.g. recombinant production) of a polypeptide or a protein of interest in a eukaryotic production host cell; and
wherein the addition of a glycosidase of step (ii) is performed by that the production host cell used in step (i) in addition to the polypeptide/protein of interest also expresses a glycosidase enzyme (such as e.g. Endo-H) whereby the initially glycosylated polypeptide of interest is deglycosylated intracellularly or following secretion and one thereby may obtain the aqueous load medium of step (ii).

It may be preferred that that the addition a glycosidase in step (ii) of the first aspect is done by adding in vitro an active glycosidase.

As discussed above—the term “glycosidase” (also called glycoside hydrolase) refers to an enzyme that catalyzes the hydrolysis of the glycosidic linkage/bond—a glycosidic bond is a type of covalent bond that joins a carbohydrate (sugar) molecule to another group, which may or may not be another carbohydrate.

As described above—a glycosidase may herein also be termed a deglycosylation enzyme.

The glycosidase may be a natural glycosidase or it may be a variant/mutated of a natural glycosidase—as known to the skilled person, one may make mutated variants of a enzyme of interest (here a glycosidase) to e.g. improve the stability of the enzyme while maintaining the key enzymatic activity (here glycosidase activity) of the enzyme.

The skilled person may know or routinely be able to determine of a polypeptide/protein of interest comprises herein relevant N-linked or O-linked glycosylation—and thereby routinely be able to determine whether or not it would in the present context be preferred to use a N-linked or O-linked glycosidase.

A number of herein e.g. commercial relevant proteins comprise N-linked glycosylation—accordingly it may be preferred to use N-linked glycosidase.

Accordingly, it may be preferred that the glycosidase used in step (ii) is a N-linked glycosidase.

As discussed above, the term N-linked glycosidase is a well-defined term in the art and the skilled person knows if a specific glycosidase of interests is a N-linked glycosidase or not.

Further the prior art describes a number of different herein suitable N-linked glycosidases.

Examples of a herein suitable N-linked glycosidase may be at least one glycosidase selected from the group consisting of: Peptide-N(4)-(N-acetyl-beta-glucosaminyl)asparagine amidase (EC number: 3.5.1.52; alternative names: N-Glycosidase-F or PNGase-F) and Endo-β-N-acetylglucosaminidase H (EC number: 3.2.1.96; alternative name ENDO-H).

The immediately above described N-linked glycosidases may in the present context be described as glycosidases that have N-linked glycosidase activity and no herein significant O-linked glycosidase activity.

Accordingly, it may herein be preferred that the N-linked glycosidase is an N-linked glycosidase that have no herein relevant O-linked glycosidase activity (such as no O-linked glycosidase activity).

The N-Glycosidase-F, also known as PNGase-F, is an asparagine amidase (EC 3.5.1.52) that may be derived from Flavobacterium mesingosepticum. It catalyses the complete and intact cleavage of N-linked oligosaccaharides from glycoproteins. It may be derived as a commercial product from New England Biolabs Inc. under the name PNGase-F or produced recombinantly in a strain like Escherichia coli.

Endo-β-N-acetylglucosaminidase H (EC 3.2.1.96), also known as ENDO-H, may be derived from Streptomyces plicatus. ENDO-H catalyses the hydrolysis of the glycosidic bond between the two N-acetylglycosamines of N-linked glycosylations. It may be derived as a commercial product from New England Biolabs Inc. under the name ENDO-H.

In particular when the enzyme of interest is a milk-clotting enzyme (e.g. selected from the group consisting of chymosin (EC 3.4.23.4), pepsin (EC 3.4.23.1) and mucorpepsin (EC 3.4.23.23))—it may be preferred that the N-linked glycosidase is Endo-β-N-acetylglucosaminidase H (EC number: 3.2.1.96; alternative name ENDO-H).

Use of ENDO-H as N-linked glycosidase in step (ii) may be particular preferred when the enzyme of interest is a mucorpepsin derived from Rhizomucor miehei (see above) or when enzyme of interest is camel chymosin (e.g. from Camelius dromedaries—see above).

As discussed above, the term O-linked glycosidase is a well-defined term in the art and the skilled person knows if a specific glycosidase of interests is a O-linked glycosidase or not.

Further the prior art describes a number of different herein suitable O-linked glycosidases.

Examples of a herein suitable O-linked glycosidase may be at least one glycosidase selected from the group consisting of: α-N-acetyl-galactosaminidase (EC number:

3.2.1.49; alternative name: GaINAC); α-galactosidase (EC number: 3.2.1.22); and neuraminidase (EC number: 3.2.1.18).

GaINAC is a highly specific exoglycosidase that catalyzes the hydrolysis of α-linked D-N-acetyl-galactosamine residues from. It may be derived as a commercial product from New England Biolabs Inc.

The effective amount/activity of a glycosidase is herein determined according to the art.

According to the art—for a N-linked glycosidase (such as e.g. PNGase-F and Endo-H) one activity unit is defined as the amount of enzyme required to remove >95% of the carbohydrate from 10 μg of denatured RNase-B in 1 hour at 37° C. in a total reaction volume of 10 μl.

For GaINAC (an O-linked glycosidase) one activity unit is defined as the amount of enzyme required to cleave >95% of the terminal α-D-N-acetyl-galactosamine from 1 nmol (GaINAcα1-3)(Fucα1-2)Galα1-4Glc-7-amino-4-methyl-coumarin (AMC), in 1 hour at 37° C. in a total reaction volume of 10 μl.

A number of herein relevant glycosidase enzymes are commercially available from e.g. the company New England Biolabs—reference is also made to the product catalogue New England Biolabs (as e.g. available on-line on their web-page) for further details in relation to specific standard definitions of herein relevant glycosidase activity units.

In relation to step (ii) relating to the addition of glycosidase to the sample of step (i)—it is herein believed that addition of from 0.001 glycosidase activity units per μg polypeptide/protein in the sample to 1000 glycosidase activity units per μg polypeptide/protein in the sample is enough to get a herein relevant deglycosylation the polypetide or protein of interest in step (ii).

If relevant for a specific purpose—one may add more glycosidase e.g. up to 20000 glycosidase activity units per pg polypeptide/protein in the sample.

As discussed above—the effective amount/activity of a glycosidase is herein determined according to the art.

### Step (iii) of First Aspect—Applying Load Material of Onto Ligands

Step (iii) of first aspect relates to applying the load medium of step (ii) onto a solid phase comprising a solid base matrix containing ligands which comprise a hydrophobic part and/or a positively charged part in order to obtain adsorption of the polypeptide of interest to the ligand.

Preferably, the solid base matrix of step (iii) is a solid base matrix containing ligands which comprise a hydrophobic part.

The term “solid base matrix” refers to the solid backbone material which contains reactive functionality permitting covalent attachment of the ligand to said backbone material. This term may herein also be referred to as solid support matrix.

As known in the art—the backbone material may be inorganic such as e. g. silica, or organic. Organic backbone materials which are useful herein include as examples cellulose and derivatives hereof, agarose, dextran, polymers such as e. g. polyacrylates, polystyrene, polyacrylamide, polymethacrylate, copolymers. Additionally, ter-and higher polymers can be used provided that at least one of the monomers contains a reactive functionality in the resulting polymer.

As known in the art—an example of a solid base matrix may be a so-called resin—as known in the art this term may be used in relation to ion-exchange chromatography (IEC).

As known in the art—the solid base matrix may preferably be particles—for instance solid base matrix may comprises particles with a particle size of less than 750 μm or particles with a particle size of less 100 μm.

Reactive functionalities of the solid support matrix permitting covalent attachment of the ligand group are well known in the art and include e. g. hydroxyl, carboxyl, thiol and amino.

As used herein, the term “ligand” refers to a group/part consisting of a hydrophobic part and/or a positively charged part as defined herein, and a spacer arm for covalently attaching the ligand to the solid base matrix. The spacer arm can be any group or substituent which is capable of covalently attaching the selected group/part to the solid base matrix. Such spacer arms are well known in the art and include e.g. alkylen groups, aromatic groups, alkylaromatic groups, amido groups, amino groups, urea groups, carbamate groups.

The aqueous load medium comprising polypeptide of interest is contacted with the ligands as described herein under conditions permitting the polypeptide of interest to bind/adsorb to the ligands. The skilled person knows how to adjust the conditions (e.g. adjust the pH such as in the range of 3-10 including the range of 4-8 and/or adjust the flow rate) in order to obtain proper adsorption of a polypeptide/protein of interest to a ligand of interest.

As such this step (iii) is a routine step for the skilled person to perform and the skilled person knows a number of different herein relevant ligands (see e.g. the review article: Yang et al, Journal of Chromatography A, 1218 (2011) 8813-8825).

Further—the skilled person knows a number of herein relevant purification/separation techniques, wherein one apply a herein relevant medium comprising polypeptide/protein of interest onto a solid phase comprising a solid base matrix containing herein relevant ligands to obtain adsorption of the polypeptide/protein of interest to the ligand.

Accordingly, the method of the first aspect or embodiments thereof as described herein may e.g. be a method, wherein the step (iii) and step (iv) are performed by use of at least one purification technique selected from the group consisting of: chromatography, column chromatography, bed adsorption, expanded bed adsorption (EBA), batch adsorption, membrane adsorption and ion-exchange chromatography (IEC).

It may be preferred that method of the first aspect or embodiments thereof as described herein is a method, wherein the step (iii) and step (iv) are performed by use of expanded bed adsorption (EBA) purification technique.

All these purification techniques are very well known to the skilled person—accordingly it is routine work for the skilled person to properly perform the steps (iii) and (iv) in relation to a specific polypeptide/protein of interest and a specific suitable used ligand.

Said in other words—it is routine work for the skilled person to identify suitable solvent, buffers etc. in order to get proper adsorption of the polypeptide of interest to the ligand in step (iii) and proper eluting the polypeptide of interest in step (iv).

Accordingly, it is not believed necessary to describe these steps in many details herein.

As known in the art—the term “chromatography” relates to a physical method of separation in which the components to be separated are distributed between two phases, one of which is termed stationary (stationary phase) while the other (the mobile phase) moves in a definite direction.

As known in the art—the term “column chromatography” relates to a separation technique in which the stationary bed is within a tube. The particles of the solid stationary phase or the support coated with a liquid stationary phase may fill the whole inside volume of the tube (packed column) or be concentrated on or along the inside tube wall leaving an open, unrestricted path for the mobile phase in the middle part of the tube (open tubular column).

As known in the art—the term “expanded bed adsorption (EBA)” relates to a preparative chromatographic technique which makes processing of viscous and particulate liquids possible.

The protein binding principles in EBA are the same as in classical column chromatography and the common ion-exchange, hydrophobic interaction and affinity chromatography ligands can be used. Where classical column chromatography uses a solid phase made by a packed bed, EBA uses particles in a fluidized state. The EBA resin contains particles of varying size and density which results in a gradient of particle size when expanded and when the bed is in its expanded state, local loops are formed. Particles such as whole cells or cell debris, which may clog a packed bed column, readily pass through a fluidized bed. EBA can therefore be used on crude culture broths or slurries of broken cells, thereby bypassing initial clearing steps such as centrifugation and filtration, which is may be required when packed beds are used.

The terms “bed adsorption”, “batch adsorption” and “membrane adsorption” are all well-known and clear to the skilled person in the present context.

As known in the art—a hydrophobic part of a ligand may e.g. be an aliphatic group or an aromatic group.

Aliphatic group may e.g. be an alkyl group with different lengths e.g. a C₂ to C₄₀ alkyl group or a C₄ to C₃₀ alkyl group;

an alkenyl group with different lengths e.g. a C_{2 to C}₄₀ alkenyl group or a C₄ to C₃₀ alkenyl group or e.g.

an alkynyl group with different lengths e.g. a C₂ to C₄₀ alkynyl group or a C₄ to C₄₀ alkynyl group.

Aromatic group may e.g. be a phenyl group or a benzyl group.

In a preferred embodiment the hydrophobic part of the ligand is a benzyl group or a phenyl group.

As known in the art—a positively charged part of a ligand may e.g. be an amino group or e.g. a quaternary ammonium group.

As known in the art—anion exchange resins have a positive charge and are used to retain and separate negatively charged compounds.

Accordingly, a solid phase comprising resin containing ligands which comprise a positively charged part of step (iii) of the method of the first aspect herein may be said to be an anion exchange resin.

Consequently, a positively charged part of a ligand may e.g. be an amino group for anion exchange or a quaternary ammonium group for anion exchange.

In a preferred embodiment the ligands comprise a hydrophobic part and a positively charged part.

Preferably—the hydrophobic part is a benzyl group and the positively charged part is an amino group—i.e. the ligand is benzylamine.

As known in the art—Mixed-mode chromatography (MMC) is a type of chromatographic method in which multiple interaction modes take place between the stationary phase and solutes in the feed. MMC is not a novel concept. For example, hydrophobic interactions have been observed in ion exchange chromatography (IEC) and affinity chromatography (AFC). Electrostatic effects often exist in size exclusion chromatography (SEC). MMC is distinctly different from single-mode chromatography because the second interaction cannot be too weak, and the two interactions should both contribute to the retention of the solutes (the term “solute” refers to the sample components in chromatography—such as e.g. the components of the load medium of step (ii) herein). For further MMC related information reference is made to e.g. the review article: Yang et al, Journal of Chromatography A, 1218 (2011) 8813-8825 with the title “Mixed-mode chromatography and its applications to biopolymers”.

Accordingly, when the ligands comprise a hydrophobic part and a positively charged part the method as described herein is preferably a method, wherein the step (iii) and step (iv) are performed by use of a mixed-mode chromatography technique.

### Step (iv) of First Aspect—Applying Load Material of Onto Ligands

Step (iv) of first aspect relates to eluting the polypeptide of interest from the solid phase in order to recover the polypeptide of interest and thereby obtaining the purified polypeptide of interest.

As discussed above—as it is routine work for the skilled person to properly perform the steps (iii) and (iv) in relation to a specific polypeptide/protein of interest and a specific suitable used ligand.

Said in other words—it is routine work for the skilled person to identify suitable solvent, buffers etc. in order to get proper adsorption of the polypeptide of interest to the ligand in step (iii) and proper eluting the polypeptide of interest in step (iv).

Accordingly, it is not believed necessary to describe e.g. step (iv) in many details herein.

The purified polypeptide of interest of step (iv) may be a composition wherein the majority of the purified polypeptide is in a not glycosylated form.

Alternatively, the purified polypeptide of interest of step (iv) may be a composition wherein e.g. less than 50% of the purified polypeptide is in a not glycosylated form.

### Separate Aspects of the Invention—Deglycosylation of Camel Chymosin as Such

As discussed in working example below—deglycosylation (preferably by use of Endo-H) of camel chymosin may improve the milk clotting activity by up to around 10-15%.

Accordingly, a separate aspect of the invention relates to a composition comprising at least 1 gram (weight dry matter) of a chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein at least 85% (w/w) of the chymosin molecules in the composition is not glycosylated in position Asn349 of SEQ ID NO: 1.

Preferably, at least 90% (w/w) of the chymosin in the composition is not glycosylated in position Asn349 of SEQ ID NO: 1, more preferably at least 95% (w/w) of the chymosin in the composition is not glycosylated in position Asn349 of SEQ ID NO: 1 and even more preferably at least 98% (w/w) of the chymosin in the composition is not glycosylated in position Asn349 of SEQ ID NO: 1.

Further, a separate aspect of the invention relates to a composition comprising at least 1 gram (weight dry matter) of a chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein at least 85% (w/w) of the chymosin molecules in the composition has Mass Spec peak below 38.5 KDa.

Preferably, at least 90% (w/w) of the chymosin in the composition has Mass Spec peak below 38.5 KDa, more preferably at least 98% (w/w) of the chymosin in the composition has Mass Spec peak below 38.5 KDa.

Further, a separate aspect of the invention relates to a method for obtaining a composition comprising at least 1 gram (weight dry matter) of a deglycosylated active chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein the method comprises the steps of:

(A): adding an active N-linked glycosidase to a sample consisting of a number of components including at least 1 g (weight dry matter) of the chymosin in a glycosylated form in order to deglycosylate the chymosin;

(B): obtaining (e.g. by purification) the deglycosylated sample of step (A) to get a composition comprising at least 1 gram (weight dry matter) of the deglycosylated active chymosin; and

wherein the activity of the deglycosylated active chymosin (IMCU/mg) obtained in step (B) is at least 5% increased as compared to an identically performed comparative method for obtaining the chymosin, which does not comprise the step (A).

Preferably, the glycosidase in step (A) is Peptide-N(4)-(N-acetyl-beta-glucosaminyl)asparagine amidase (EC number: 3.5.1.52; alternative names: N-Glycosidase-F or PNGase-F) or Endo-β-N-acetylglucosaminidase H (EC number: 3.2.1.96; alternative name ENDO-H).

More preferably glycosidase in step (A) is Endo-β-N-acetylglucosaminidase H (EC number: 3.2.1.96; alternative name ENDO-H).

It is routine work for the skilled person to obtain a sample consisting of a number of components including at least 1 g (weight dry matter) of the chymosin in a glycosylated form of step (A).

It may e.g. be obtained by production (e.g. recombinant production) of e.g. a protein of interest in a eukaryotic production host cell, such as e.g. a fungal cells (such as e.g. Aspergillus cells—preferably Aspergillus niger or Aspergillus oryzae).

As understood by the skilled person in the art—a composition comprising the obtained deglycosylated active chymosin of step (B) would have a different glycosylation profile—i.e. it could be considered a new composition as such.

Accordingly, a further separate aspect of the invention relates to a composition comprising deglycosylated active chymosin, wherein the composition is obtainable by the method for obtaining a composition comprising at least 1 gram (weight dry matter) of a deglycosylated active chymosin of the separate aspect above and embodiments thereof as described herein.

For all the separate aspects and embodiments thereof immediately above—preferably the chymosin comprises the mature polypeptide of SEQ ID NO: 1 (Camel chymosin), which is from amino acid position 59 to amino acid position 381 of SEQ ID NO: 1.

In relation to these separate aspects immediately above—below discussed prior art may be of relevance.

Amongst the aspartic proteases it has been shown that deglycosylation of mucorpepsin derived from Rhizomucor miehei may improve the milk clotting activity by up to around 25% (EP0805866B1/U.S. Pat. No. 6,127,142A; Harboe et al, Chr. Hansen A/S, Denmark).

THERMOLASE® is a commercial available (from Chr. Hansen A/S; Denmark) milk-clotting enzyme, produced by fermentation of a selected strain of the fungus Cryphonectria parasitica.

CHY-MAX® is a commercial available (from Chr. Hansen A/S; Denmark) milk-clotting enzyme from bovine.

Not published results of the applicant of the present invention demonstrated that deglycosylation of THERMOLASE® or CHY-MAX® did not result in significant improved milk clotting activity—i.e. as would be expected by the skilled person—it is not all enzymes that get improved activity after deglycosylation.

WO02/36752A2 (Chr. Hansen A/S, Denmark) describes recombinant production of so-called non-bovine chymosin (such as e.g. from ovine, caprine camel, buffalo or Lama). On page 13 is described in general terms that such a non-bovine chymosin may be deglycosylated. However, WO02/36752A2 provides no herein relevant experimental data in relation to deglycosylation of the mentioned so-called non-bovine chymosins (such as e.g. camel chymosin).

### Aspects/Embodiments Herein—Presented in Claim Format

Herein described aspects and preferred embodiments of the invention may be presented/described in a so-called claim format—this is done below.

1. A method for purifying a polypeptide of interest from an aqueous medium comprising such a polypeptide of interest, wherein the method comprises the steps of:

(i): obtaining an aqueous sample consisting of a number of components including the polypeptide of interest in a glycosylated form;

(ii): adding a glycosidase and/or a chemical treatment (such as such as treatment with periodate) to the sample of step (i) in order to deglycosylate the polypeptide of interest to obtain an aqueous load medium;

(iii): applying the load medium of step (ii) onto a solid phase comprising a solid base matrix containing ligands which comprise a hydrophobic part and/or a positively charged part in order to obtain adsorption of the polypeptide of interest to the ligand;

(iv): eluting the polypeptide of interest from the solid phase in order to recover the polypeptide of interest and thereby obtaining the purified polypeptide of interest;

wherein the amount of the purified polypeptide of interest (number of molecules) obtained in step (iv) is at least 5% increased as compared to an identically performed comparative method for purifying the polypeptide of interest, which does not comprise the step (ii).

2. The method of claim 1, wherein there in step (ii) is added a glycosidase.

3. The method of claim 1, wherein the addition a glycosidase in step (ii) of claim 1 is done by adding in vitro an active glycosidase.

4. The method of any of the preceding claims, wherein the amount of the polypeptide of interest (number of molecules) obtained in step (iv) is at least 50% increased as compared to an identically performed comparative method for purifying the polypeptide of interest, which does not comprise the step (ii).

5. The method of any of the preceding claims, wherein the polypeptide of interest is a protein of interest.

6. The method of claim 5, wherein the protein of interest is an enzyme of interest.

7. The method of claim 6, wherein the enzyme of interest is a milk-clotting enzyme of interest.

8. The method of claim 6, wherein the milk-clotting enzyme of interest is a milk-clotting enzyme selected from the group consisting of chymosin (EC 3.4.23.4), pepsin (EC 3.4.23.1) and mucorpepsin (EC 3.4.23.23).

9. The method of claim 8, wherein the milk-clotting enzyme of interest is:

mucorpepsin derived from Rhizomucor miehei; or

a chymosin, wherein the polypeptide sequence of the chymosin comprises a sequence, which has at least 90% sequence identity with the mature polypeptide of SEQ ID NO: 1 (Camel chymosin), which is from amino acid position 59 to amino acid position 381 of SEQ ID NO: 1.

10. The method of claim 9, wherein the milk-clotting enzyme of interest is a chymosin, wherein the polypeptide sequence of the chymosin comprises a sequence, which has at least 99% sequence identity with the mature polypeptide of SEQ ID NO: 1 (Camel chymosin), which is from amino acid position 59 to amino acid position 381 of SEQ ID NO: 1.

11. The method of any of the preceding claims, wherein the sample consisting of a number of components including the polypetide/protein of interest in a glycosylated form as required in step (i) of claim 1 is obtained by production (e.g. recombinant production) of a polypeptide or a protein of interest in a eukaryotic production host cell.

12. The method of claim 11, wherein the eukaryotic production host cell is an Aspergillus cell, preferably Aspergillus niger or Aspergillus oryzae.

13. The method of claim 11, wherein the protein of interest is milk-clotting enzyme of interest and the milk-clotting enzyme of interest is:

mucorpepsin derived from Rhizomucor miehei and the eukaryotic production host cell is Rhizomucor miehei; or

a chymosin, wherein the polypeptide sequence of the chymosin comprises a sequence, which has at least 90% sequence identity with the mature polypeptide of SEQ ID NO: 1 (Camel chymosin), which is from amino acid position 59 to amino acid position 381 of SEQ ID NO: 1 and the eukaryotic production host cell is an Aspergillus cell, preferably Aspergillus niger or Aspergillus oryzae.

14. The method of any of the preceding claims, wherein the glycosidase used in step (ii) is a N-linked glycosidase.

15. The method of claim 14, wherein the N-linked glycosidase is at least one glycosidase selected from the group consisting of: Peptide-N(4)-(N-acetyl-beta-glucosaminyl)asparagine amidase (EC number: 3.5.1.52; alternative names: N-Glycosidase-F or PNGase-F) and Endo-β-N-acetylglucosaminidase H (EC number: 3.2.1.96; alternative name ENDO-H).

16. The method of claim 15, wherein the N-linked glycosidase is Endo-β-N-acetylglucosaminidase H (EC number: 3.2.1.96; alternative name ENDO-H) and the protein of interest is milk-clotting enzyme of interest and the milk-clotting enzyme of interest is:

mucorpepsin derived from Rhizomucor miehei; or

a chymosin, wherein the polypeptide sequence of the chymosin comprises a sequence, which has at least 90% sequence identity with the mature polypeptide of SEQ ID NO: 1 (Camel chymosin), which is from amino acid position 59 to amino acid position 381 of SEQ ID NO: 1.

17. The method of any of the preceding claims, wherein the solid base matrix comprises particles with a particle size of less than 750 μm and wherein the solid base matrix is made from at least one of following materials: silica, cellulose, agarose, dextran, polyacrylates, polystyrene, polyacrylamide or polymethacrylate.

18. The method of any of the preceding claims, wherein the step (iii) and step (iv) are performed by use of at least one purification technique selected from the group consisting of: chromatography, column chromatography, bed adsorption, expanded bed adsorption (EBA), batch adsorption, membrane adsorption and ion-exchange chromatography (IEC).

19. The method of claim 18, wherein the step (iii) and step (iv) are performed by use of expanded bed adsorption (EBA) purification technique.

20. The method of any of the preceding claims, wherein the ligands comprise a hydrophobic part and wherein the hydrophobic part of the ligand is an aliphatic group or an aromatic group.

21. The method of claim 20, wherein the aliphatic group is:

a C₂ to C₄₀ alkyl group;

a C₂ to C₄₀ alkenyl group or

a C₂ to C₄₀ alkynyl group;

or

wherein the aromatic group is a phenyl group or a benzyl group.

22. The method of claim 21, hydrophobic part of the ligand is an aromatic group, which is a benzyl group.

23. The method of any of the preceding claims, wherein the ligands comprise a positively charged part and wherein the positively charged part of the ligand is an amino group.

24. The method of any of the preceding claims, wherein the ligands comprise a hydrophobic part and a positively charged part.

25. The method of claim 24, wherein the ligand is benzylamine.

26. The method of any of the claims 24 to 25, wherein the step (iii) and step (iv) are performed by use of Mixed-mode chromatography (MMC) purification technique, preferably wherein Mixed-mode chromatography (MMC) purification technique is expanded bed adsorption (EBA) technique.

27. The method of any of the claims 24 to 26, wherein protein of interest is a milk-clotting enzyme of interest.

28. The method of claim 27, wherein the milk-clotting enzyme of interest is a milk-clotting enzyme selected from the group consisting of chymosin (EC 3.4.23.4), pepsin (EC 3.4.23.1) and mucorpepsin (EC 3.4.23.23).

29. The method of claim 28, wherein the milk-clotting enzyme of interest is:

mucorpepsin derived from Rhizomucor miehei; or

a chymosin, wherein the polypeptide sequence of the chymosin comprises a sequence, which has at least 90% sequence identity with the mature polypeptide of SEQ ID NO: 1 (Camel chymosin), which is from amino acid position 59 to amino acid position 381 of SEQ ID NO: 1.

30. The method of claim 29, wherein the ligand is benzylamine.

31. A composition comprising purified polypeptide of interest, wherein the composition is obtainable by the method of any of the claims 1 to 31.

32. A method for purifying a milk clotting enzyme of interest from an aqueous medium comprising such a milk clotting enzyme of interest, wherein the method comprises the steps of:

(I): producing milk clotting enzyme of interest in a production host cell, wherein the production host cell does not give significant glycosylation of the milk clotting enzyme, to obtain an aqueous sample consisting of a number of components including at least 10 gram (weight dry matter) (such as preferably at least 1 kg weight dry matter) of the milk clotting enzyme of interest in an essentially not glycosylated form and thereby obtaining an aqueous load medium;

(II): applying the load medium of step (I) onto a solid phase comprising a solid base matrix containing ligands which comprise a hydrophobic part and/or a positively charged part in order to obtain adsorption of the polypeptide of interest to the ligand; and

(III): eluting the milk clotting enzyme of interest from the solid phase in order to recover the milk clotting enzyme of interest and thereby obtaining the purified milk clotting enzyme of interest.

33. The method of claim 32, wherein the production host cell of step (I) is a prokaryotic production host cell (such as e.g. E. coli of Bacillus).

34. The method of claim 32, wherein the production host cell of step (I) is an eukaryotic production host cell that does not give significant glycosylation of the milk clotting enzyme of interest.

35. The method of claim 34 wherein the eukaryotic production host cell is a genetically engineered cell, wherein genes essential for glycosylated are inactivated (e.g. deleted or mutated).

36. The method of any of the claims 32 to 35, wherein the ligands comprise a hydrophobic part and wherein the hydrophobic part of the ligand is an aliphatic group or an aromatic group.

37. The method of claim 36, wherein the aliphatic group is:

a C₂ to C₄₀ alkyl group;

a C₂ to C₄₀ alkenyl group or

a C₂ to C₄₀ alkynyl group;

or

wherein the aromatic group is a phenyl group or a benzyl group.

38. The method of claim 37, hydrophobic part of the ligand is an aromatic group, which is a benzyl group.

39. The method of any of the claims 32 to 38, wherein the ligands comprise a positively charged part and wherein the positively charged part of the ligand is an amino group.

40. The method of any of the claims 32 to 39, wherein the ligands comprise a hydrophobic part and a positively charged part.

41. The method of claim 40, wherein the ligand is benzylamine.

42. The method of any of the claims 40 to 41, wherein the step (iii) and step (iv) are performed by use of Mixed-mode chromatography (MMC) purification technique, preferably wherein Mixed-mode chromatography (MMC) purification technique is expanded bed adsorption (EBA) technique.

43. The method of any of the claims 32 to 42, wherein the milk-clotting enzyme of interest is a milk-clotting enzyme selected from the group consisting of chymosin (EC 3.4.23.4), pepsin (EC 3.4.23.1) and mucorpepsin (EC 3.4.23.23).

44. The method of claim 43, wherein the milk-clotting enzyme of interest is:

mucorpepsin derived from Rhizomucor miehei; or

a chymosin, wherein the polypeptide sequence of the chymosin comprises a sequence, which has at least 90% sequence identity with the mature polypeptide of SEQ ID NO:

1 (Camel chymosin), which is from amino acid position 59 to amino acid position 381 of SEQ ID NO: 1.

45. The method of claim 44, wherein the ligand is benzylamine.

46. A composition comprising at least 1 gram (weight dry matter) of a chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein at least 85% (w/w) of the chymosin molecules in the composition is not glycosylated in position Asn349 of SEQ ID NO: 1.

47. The composition of claim 46, wherein at least 90% (w/w) of the chymosin in the composition is not glycosylated in position Asn349 of SEQ ID NO: 1, more preferably at least 95% (w/w) of the chymosin in the composition is not glycosylated in position Asn349 of SEQ ID NO: 1 and even more preferably at least 98% (w/w) of the chymosin in the composition is not glycosylated in position Asn349 of SEQ ID NO: 1.

48. A composition comprising at least 1 gram (weight dry matter) of a chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein at least 85% (w/w) of the chymosin molecules in the composition has Mass Spec peak below 38.5 KDa.

49. The composition of claim 48, wherein at least 90% (w/w) of the chymosin in the composition has Mass Spec peak below 38.5 KDa, more preferably at least 98% (w/w) of the chymosin in the composition has Mass Spec peak below 38.5 KDa.

50. A method for obtaining a composition comprising at least 1 gram (weight dry matter) of a deglycosylated active chymosin, wherein the polypeptide sequence of the chymosin comprises a sequence, which has at least 95% sequence identity with the mature polypeptide of SEQ ID NO: 1 (Camel chymosin), which is from amino acid position 59 to amino acid position 381 of SEQ ID NO: 1, wherein the method comprises the steps of:

(A): adding an active N-linked glycosidase to a sample consisting of a number of components including at least 1 g (weight dry matter) of the chymosin in a glycosylated form in order to deglycosylate the chymosin;

(B): obtaining (e.g. by purification) the deglycosylated sample of step (A) to get a composition comprising at least 1 gram (weight dry matter) of the deglycosylated active chymosin; and

wherein the activity of the deglycosylated active chymosin (IMCU/mg) obtained in step (B) is at least 5% increased as compared to an identically performed comparative method for obtaining the chymosin, which does not comprise the step (A).

51. The method of claim 50, wherein the glycosidase in step (A) is Peptide-N(4)-(N-acetyl-beta-glucosaminyl)asparagine amidase (EC number: 3.5.1.52; alternative names: N-Glycosidase-F or PNGase-F) or Endo-β-N-acetylglucosaminidase H (EC number: 3.2.1.96; alternative name ENDO-H).

52. The method of claim 50, wherein the glycosidase in step (A) is Endo-β-N-acetylglucosaminidase H (EC number: 3.2.1.96; alternative name ENDO-H).

53. The method of any of the claims 50 to 52, the sample consisting of a number of components including the chymosin in a glycosylated form as required in step (A) of claim 50 is obtained by production (e.g. recombinant production) of a polypeptide or a protein of interest in a eukaryotic production host cell.

54. The method of claim 53, wherein the eukaryotic production host cell is a fungal cells (such as e.g. Aspergillus cells—preferably Aspergillus niger or Aspergillus oryzae).

55. The method of any of the claims 50 to 54, wherein the chymosin comprises the mature polypeptide of SEQ ID NO: 1 (Camel chymosin), which is from amino acid position 59 to amino acid position 381 of SEQ ID NO: 1.

56. A composition comprising deglycosylated active chymosin, wherein the composition is obtainable by the method for obtaining a composition comprising at least 1 gram (weight dry matter) of a deglycosylated active chymosin of any of the claims 50 to 55.

57. The composition of claim 56, wherein the chymosin comprises the mature polypeptide of SEQ ID NO: 1 (Camel chymosin), which is from amino acid position 59 to amino acid position 381 of SEQ ID NO: 1.

### EXAMPLES

### Example 1

### Improved Purification of Milk-Clotting Enzymes via a Deglycosylation Step

Materials:

Step (i): Samples with the Polypeptide of Interest in a Glycosylated Form

(a): Mucorpepsin derived from Rhizomucor miehei as described in e.g. EP0805866B1 (Harboe et al, Chr. Hansen A/S, Denmark). It was produced using Rhizomucor miehei as production host cell.

(b): Recombinant produced (production host cells was Aspergillus niger) Camelius dromedarius chymosin as described in e.g. WO02/36752A2 (Chr. Hansen). It may herein alternatively be termed camel chymosin and the publically known amino acid sequence is shown in SEQ ID NO: 1 herein.

Both samples (a) and (b) were a so-called first filtrate—i.e. before the further down-stream purification was removed/separated production host cells and other unwanted material in the fermentation media by e.g. centrifugation and/or filtrating.

Step (ii): Adding a Glycosidase

The glycosidase used was ENDO-H.

Step (iii): Solid Base Matrix Containing Ligands

The ligand was benzylamine covalently bound to agarose solid base matrix particles. It may in this example be termed resin.

This ligand is an example of a ligand which comprises a hydrophobic part and/or a positively charged part.

Experiments:

For each of the samples (i.e. mucorpepsin and camel chymosin) was made two expanded bed adsorption (EBA) purifications experiments—one performed according to the method of the first aspect as described herein (i.e. with the addition of a glycosidase step (ii)) and another comparative experiment where everything was completely identically performed expect that the adding a glycosidase step (ii) was not used in the comparative experiment.

Results:

Determined as milk-clotting activity (C) expressed in International Milk-Clotting Units (IMCU) per ml resin—for the mucorpepsin was there around 50% improved binding to the ligand due to the use of the glycosidase step (ii)—i.e. 50% more activity (IMCU/ml resin) as compared to the comparative experiment where glycosidase step (ii) was not used.

As discussed herein—EP0805866B1/U.S. Pat. No. 6,127,142A; Harboe et al, Chr. Hansen A/S, Denmark) describes that deglycosylation of mucorpepsin derived from Rhizomucor miehei may improve the milk clotting activity by up to around 25%.

Accordingly, use of the glycosidase step (ii) of the method of the first aspect seems to have increased the amount of the purified mucorpepsin (number of molecules) by at least 25%.

Determined as milk-clotting activity (C) expressed in International Milk-Clotting Units (IMCU) per ml resin—for the camel chymosin was there around 80% improved binding to the ligand due to the use of the glycosidase step (ii)—i.e. 80% more activity (IMCU/ml resin) as compared to the comparative experiment where glycosidase step (ii) was not used.

As discussed herein—Endo-H deglycosylation of camel chymosin may improve the milk clotting activity by up to around 10-15%.

Accordingly, use of the glycosidase step (ii) of the method of the first aspect seems to have increased the amount of the purified camel chymosin (number of molecules) by at least 65%.

### Conclusions

The results above demonstrated that:
use of the glycosidase step (ii) of the method of the first aspect seems to have increased the amount of the purified mucorpepsin (number of molecules) by at least 25% and
use of the glycosidase step (ii) of the method of the first aspect seems to have increased the amount of the purified camel chymosin (number of molecules) by at least 65%.

### Example 2

### Deglycosylation of Camel Chymosin—Improved activity

Recombinant produced (production host cells was Aspergillus niger—as described in e.g. WO02/36752A2, Chr. Hansen) Camelius dromedarius chymosin were analyzed. It may herein alternatively be termed camel chymosin and the publically known amino acid sequence is shown in SEQ ID NO: 1 herein.

The sequence of camel chymosin suggests two potential glycosylation sites, Asn158 and Asn349 of SEQ ID NO: 1.

Six variants of camel chymosin were separated by hydrophobic interaction chromatography.

They were characterized with mass spectrometry, SDS-PAGE, milk-clotting assay, N-terminal sequencing, and deglycosylation.

The protein mass was measured using a Voyager Elite MALDI TOF mass spectrometer (Applied Biosystems Inc., Framingham, MA) operated in linear, positive ion mode. The separated variants were desalted and concentrated on 50R1 micro columns, subsequently eluted and deposited on a stainless steel MALDI target with a matrix solution consisting of 20 mg/ml sinnapinic acid in 70% acetonitrile and 0.1% trifluoroacetic acid. The target spots were pretreated with 0.5 μl of sinnapinic acid in acetone (20 mg/ml). Samples were analyzed in the mass range of 3-50 kDa. The data were baseline-corrected and noise filtered.

Mass spectrometry, SDS-PAGE, and milk-clotting assay showed that the glycosylation and activity of the variants was:

| Variant no. | 1 | 2 | 3 | 4 | 5 | 6 |
|---|---|---|---|---|---|---|
| No. of glycosylations | 2 | 2 | 1 | 1 | 0 | 0 |
| Mass Spec peak(kDa)* | 40.2 | 40.2 | 37.7 | 37.7 | 35.6 | 35.8 |
| Milk-clotting activity | 123 | 289 | 396 | 467 | 474 | 426 |
| (IMCU/mg) | | | | | | |

*The average mass of the peaks in the spectra

During storage, degradation takes places at a site between the two glycosylation sites. The fragment containing Asn158 was found for all variants. It was glycosylated for variants 1-4, but a nonglycosylated fragment was found only for variants 5-6. This suggests that the single glycosylated variants 3+4 are glycosylated at Asn158. This indicates that the additional glycosylation of Asn349 (for variants 1+2) is responsible for the observed decrease in milk-clotting activity. Thus a deglycosylation of camel chymosin should cause an increase in the activity.

Deglycosylation of the total sample comprising all the six variants of the table above with ENDO-H gave a composition with improved Milk-clotting activity (IMCU/mg)—around 10-15% improved Milk-clotting activity (IMCU/mg).

Deglycosylation of the total sample comprising all the six variants of the table above with PNGase-F did not improve the Milk-clotting activity as good as with ENDO-H.

### Example 3

### Improved Purification of Milk-Clotting Enzymes via a Deglycosylation Step

Materials:

Step (i): Samples With the Polypeptide of Interest in a Flycosylated Form

Recombinant produced Camelius dromedarius—see Example 1 above.

Step (ii): Adding a Glycosidase

The glycosidase used was ENDO-H.

Step (iii): Solid Base Matrix Containing Ligands

The ligand was phenyl covalently bound to agarose (e.g. Superose®) solid base matrix particles.

It may in this example be termed resin.

This ligand is an example of a ligand which comprises a hydrophobic part.

Experiments:

For the camel chymosin sample two packed bed purification experiments were conducted—one performed according to the method of the first aspect as described herein (i.e. with the addition of a glycosidase step (ii)) and another comparative experiment where everything was completely identically performed expect that the adding a glycosidase step (ii) was not used in the comparative experiment.

Results:

Determined as milk-clotting activity (C) expressed in International Milk-Clotting Units (IMCU) per ml resin—for the camel chymosin was there around 75% improved binding (based on 10% break-through) to the ligand due to the use of the glycosidase step (ii)—i.e. 75% more activity (IMCU/ml resin) as compared to the comparative experiment where glycosidase step (ii) was not used.

As discussed herein—Endo-H deglycosylation of camel chymosin may improve the milk clotting activity by up to around 10-15%.

Accordingly, use of the glycosidase step (ii) of the method of the first aspect seems to have increased the amount of the purified camel chymosin (number of molecules) by at least 60%.

### Conclusions

The results above demonstrated that:
use of the glycosidase step (ii) of the method of the first aspect seems to have increased the amount of the purified camel chymosin (number of molecules) by at least 60%.

### Example 4

### Improved Purification of Milk-Clotting Enzymes via a Deglycosylation Step

Materials:

Step (i): Samples With the Polypeptide of Interest in a Glycosylated Form

Recombinant produced Camelius dromedarius—see Example 1 above.

Step (ii): Adding a Glycosidase

The glycosidase used was ENDO-H.

Step (iii): Solid Base Matrix Containing Ligands

The ligand was a quartenary amine covalently bound to agarose solid base matrix particles.

It may in this example be termed resin.

This ligand is an example of a ligand which comprises a positively charged part.

Experiments:

For the camel chymosin sample two batch adsorption purification experiments were conducted—one performed according to the method of the first aspect as described herein (i.e. with the addition of a glycosidase step (ii)) and another comparative experiment where everything was completely identically performed expect that the adding a glycosidase step (ii) was not used in the comparative experiment.

Results:

Determined as milk-clotting activity (C) expressed in International Milk-Clotting Units (IMCU) per ml resin—for the camel chymosin was there around 20% improved binding (based on static binding) to the ligand due to the use of the glycosidase step (ii)—i.e. 20% more activity (IMCU/ml resin) as compared to the comparative experiment where glycosidase step (ii) was not used.

As discussed herein—Endo-H deglycosylation of camel chymosin may improve the milk clotting activity by up to around 10-15%.

Accordingly, use of the glycosidase step (ii) of the method of the first aspect seems to have increased the amount of the purified camel chymosin (number of molecules) by at least 5%.

### Conclusions

The results above demonstrated that:
use of the glycosidase step (ii) of the method of the first aspect seems to have increased the amount of the purified camel chymosin (number of molecules) by at least 5%.

### REFERENCES

1: WO01/58924A2 (Upfront Chromatography A/S, Denmark)

2: Yang et al, Journal of Chromatography A, 1218 (2011) 8813-8825. Review article with title “Mixed-mode chromatography and its applications to biopolymers”.

3: EP0805866B1/US6127142A; Harboe et al, Chr. Hansen A/S, Denmark

4: W002/36752A2 (Chr. Hansen)

1-57. (canceled)

58. A method for purifying a milk-clotting enzyme of interest from an aqueous sample comprising such an enzyme of interest, wherein the method comprises:
(i) obtaining an aqueous sample comprising a milk-clotting enzyme of interest in a glycosylated form and other components;
(ii) deglycosylating the milk-clotting enzyme of interest to obtain an aqueous load medium;
(iii) applying the aqueous load medium of step (ii) onto a solid phase comprising a solid base matrix containing ligands which comprise a hydrophobic part and/or a positively charged part to adsorb the milk-clotting enzyme to the ligands of the solid phase; and
(iv) eluting the milk-clotting enzyme of interest from the solid phase to recover purified milk-clotting enzyme of interest;
wherein the number of molecules of purified enzyme of interest obtained in step (iv) is at least 5% greater than that obtained by a comparative method that does not include step (ii).

59. The method of claim 58, wherein step (ii) comprises adding a glycosidase to the aqueous sample of step (i).

60. The method of claim 58, wherein step (ii) comprises treating the aqueous sample of step (i) with periodate.

61. The method of claim 58, wherein the milk-clotting enzyme of interest is a milk-clotting enzyme selected from the group consisting of chymosin (EC 3.4.23.4), pepsin (EC 3.4.23.1) and mucorpepsin (EC 3.4.23.23).

62. The method of claim 61, wherein the milk-clotting enzyme of interest is selected from the group consisting of:
mucorpepsin derived from Rhizomucor miehei; and
a chymosin, wherein the polypeptide sequence of the chymosin comprises a sequence having at least 90% sequence identity to the mature polypeptide of SEQ ID NO: 1 (Camel chymosin), which is from amino acid position 59 to amino acid position 381 of SEQ ID NO: 1.

63. The method of claim 58, wherein the method further comprises obtaining the aqueous sample of step (i) by a process comprising recombinant production of the milk-clotting enzyme of interest in a eukaryotic production host cell.

64. The method of claim 63, wherein the eukaryotic production host cell is an Aspergillus cell selected from the group consisting of Aspergillus niger and Aspergillus oryzae.

65. The method of claim 59, wherein the glycosidase used in step (ii) is a N-linked glycosidase.

66. The method of claim 65, wherein the N-linked glycosidase is at least one selected from the group consisting of Peptide-N(4)-(N-acetyl-beta-glucosaminyl)asparagine amidase (EC number: 3.5.1.52; alternative names: N-Glycosidase-F or PNGase-F) and Endo-β-N-acetylglucosaminidase H (EC number: 3.2.1.96; alternative name ENDO-H).

67. The method of claim 58, wherein the solid base matrix comprises particles with a particle size of less than 750 μm and wherein the solid base matrix is made from at least one material selected from the group consisting of silica, cellulose, agarose, dextran, poly-acrylates, polystyrene, polyacrylamide and polymethacrylate.

68. The method of claim 58, wherein step (iii) and step (iv) are performed by at least one purification technique selected from the group consisting of chromatography, column chromatography, bed adsorption, expanded bed adsorption (EBA), batch adsorption, membrane adsorption and ion-exchange chromatography (IEC).

69. The method of claim 58, wherein the ligands of the solid base matrix comprise a hydrophobic part that is an aliphatic group or an aromatic group.

70. The method of claim 69, wherein the hydrophobic part of the ligands is an aliphatic group selected from the group consisting of a C2 to C40 alkyl group; a C2 to C40 alkenyl group; and a C2 to C40 alkynyl group.

71. The method of claim 69, wherein the hydrophobic part of the ligands is an aromatic group selected from the group consisting of a phenyl group and a benzyl group.

72. The method of claim 69, wherein the hydrophobic part of the ligands is a benzyl group.

73. The method of claim 58, wherein the ligands of the solid base matrix comprise a positively charged part that is an amino group.

74. The method of claim 58, wherein the ligands of the solid base matrix comprise a hydrophobic part and a positively charged part.

75. The method of claim 74, wherein the ligands comprise a benzylamine group.

76. A composition comprising a purified milk-clotting enzyme of interest obtained by the method of claim 58.
